(19) United States
(12) Patent Application Publication
(10) Pub. No.: US 2014/0255365 A1
(43) Pub. Date: Sep. 11, 2014

(54) Oral Electrolyte Solution Containing Lactoferrin and Uses Thereof

(71) Applicant: MEAD JOHNSON NUTRITION COMPANY, Glenview, IL (US)

(72) Inventors: Juan M. Gonzalez, Newburgh, IN (US); Dattatreya Banavara, Newburgh, IN (US); John Alvey, Evansville, IN (US)

(73) Assignee: Mead Johnson Nutrition Company, Glenview, IL (US)

(21) Appl. No.: 13/794,014

(22) Filed: Mar. 11, 2013

(51) Int. Cl. A61K 38/40; A23C 17/02; A61K 35/74

(52) U.S. Cl. CPC A61K 38/40; A61K 35/747; A23C 17/02
USPC 424/93.45; 514/2.5

(57) ABSTRACT

The present disclosure relates to an oral electrolyte solution comprising lactoferrin. Lactoferrin may promote intestinal healing during a bout of diarrhea in a pediatric subject, thereby reducing the duration of diarrhea symptoms. Additionally, the oral electrolyte solutions disclosed herein may comprise Lactobacillus rhamnosus GG, vitamin B₃ and/or a source of zinc. The disclosure further relates to methods of reducing the duration of diarrhea symptoms and reducing gastrointestinal irritation in a pediatric subject by providing an oral electrolyte solution comprising lactoferrin.

### TECHNICAL FIELD

The present disclosure relates generally to oral electrolyte solutions comprising lactoferrin. The oral electrolyte solutions are suitable for administration to pediatric subjects. Additionally, the disclosure relates to methods of providing lactoferrin in an oral electrolyte solution to a pediatric subject to promote fluid retention and reduce gastrointestinal symptoms of diarrhea. Further, the disclosure relates to methods of reducing the duration of diarrhea in a pediatric subject by providing an oral electrolyte solution comprising lactoferrin.

### BACKGROUND ART

Lactoferrin, an iron-binding glycoprotein, is one of the major multifunctional agents present in human milk. It has the capacity to bind two molecules of iron in a reversible fashion and can facilitate the uptake of iron within the intestines. Additionally, it is the second most abundant protein in the whey fraction of human milk. Functionally, lactoferrin regulates iron absorption and, as such, can bind iron-based free radicals as well as donate iron for supporting an immunological response. Further, lactoferrin has been shown to be both bacteriostatic and bactericidal, and it aids in preventing intestinal infections in humans, especially in pediatric subjects.

Lactoferrins are single chain polypeptides of about 80 kD containing 1-4 glycans, depending on the species. The 3-D structures of lactoferrin of different species are very similar, but not identical. Each lactoferrin comprises two homologous lobes, called the N- and C-lobes, referring to the N-terminal and C-terminal part of the molecule, respectively. Each lobe further consists of two sub-lobes or domains, which form a cleft where the ferric ion (Fe³⁺) is tightly bound in synergistic cooperation with a (bi)carbonate anion. These domains are called N1, N2, C1 and C2, respectively. The N-terminus of lactoferrin has strong cationic peptide regions that are responsible for a number of important binding characteristics. Lactoferrin has a very high isoelectric point (˜pI 9) and its cationic nature plays a major role in its ability to defend against bacterial, viral, and fungal pathogens. There are several clusters of cationic amino acids residues within the N-terminal region of lactoferrin mediating the biological activities of lactoferrin against a wide range of microorganisms.

Human lactoferrin has been reported to protect against Gram-negative bacteria in a variety of ways. It is believed that human lactoferrin exerts a bacteriostatic activity by depriving microorganisms of the iron that is necessary for growth. Thus, by sequestering the environmental iron of pathogenic microorganisms, human lactoferrin effectively inhibits the growth of those microorganisms.

Several studies have examined the effect of human lactoferrin on various bacterial species. For example, a 2001 study demonstrated that human lactoferrin can inhibit the adhesion of EPEC to HeLa cells. (Nascimento de Arujao, A., et al., Lactoferrin and Free Secretory Component of Human Milk Inhibit the Adhesion of Enteropathogenic Escherichia coli to HeLa Cells, BMC Microbiol. 1:25 (2001)).

Further, human lactoferrin appears to have a positive effect on the symptoms of diarrheal diseases, yet the addition of lactoferrin to commercially viable nutritional compositions has generally been limited due to lactoferrin's proclivity to lose functional capacity during processing steps that involve significant fluctuation in temperature and/or pH.

Without being bound by any particular theory, lactoferrin may inhibit growth and impair the virulence of some pathogens by decreasing their ability to adhere to or invade mammalian cells, and by binding to, or degrading specific virulent proteins. Additionally, lactoferrin may protect infants from sepsis by blocking attachment and invasion of organisms in the gut.

Oral electrolyte therapy has been used to promote rehydration during the early stages of diarrhea in infants and children. Oral electrolyte therapy utilizes an electrolyte solution typically formulated with potassium (K), sodium (Na), chloride (Cl), and bicarbonate (CO₃) ions to rehydrate a subject suffering from diarrhea. Often, the use of oral electrolyte therapy reduces the need for intravenous rehydration therapy in more than 80% of cases. During a bout of diarrhea, a subject may experience disturbed electrolyte metabolism. Therefore, replacement of ions such as sodium, chloride, and calcium can reduce the duration of diarrhea symptoms.

In addition to dehydration, an infant or child with diarrhea experiences an irritation in the gastrointestinal track. This irritation may be caused by the presence of a microorganism, such as a bacterium or other pathogen. When this irritation occurs, the production of enzymes and the absorption of nutrients and water throughout in intestinal mucosa is impaired. This can lead to dehydration, loss of minerals and other nutrients, and to the disruption of the infant or child's feeding pattern resulting in weight loss.

One such example of a bacterium known to cause pathogenesis is Escherichia coli which may cause diarrhea in infants, children and adults and is realized as an agent for pediatric diarrhea. As defined in “Lactoferrin Impairs Type III Secretory System Function In Enteropathogenic Escherichia Coli” as published in INFECTION AND IMMUNITY, pp 5149-5155 (2003), there are generally three stages to enteropathogenic E. coli pathogenesis. Harmful symptoms occur when bacterial proteins, such as EspB emitted by the E. coli inhibits the interaction between various myosin proteins and actin filaments in suppressing phagocytosis, leading to diarrhea or other gastric distress in infants, children and adults.

Accordingly, what is needed is an oral electrolyte solution comprising lactoferrin. The oral electrolyte solutions may reduce the duration of diarrhea when provided to a pediatric subject. Additionally, the oral electrolyte solution(s) described herein comprising lactoferrin can promote rehydration and provide a balance of electrolytes.

### BRIEF SUMMARY

Briefly, the present disclosure is directed, in an embodiment, to an oral electrolyte solution comprising lactoferrin. In certain embodiments the oral electrolyte solution may further comprise a probiotic, such as Lactobacillus rhamnosus GG (“LGG”), vitamin B₃, zinc, cultured butter milk fraction and mixtures of one or more thereof.

Additionally, the disclosure is directed to a method of reducing the duration of diarrhea in a pediatric subject. In other embodiments, the disclosure relates to methods for reducing gastrointestinal irritation and rehydrating a pediatric subject during a bout of diarrhea.

It is to be understood that both the foregoing general description and the following detailed description present embodiments of the disclosure and are intended to provide an overview or framework for understanding the nature and character of the disclosure as it is claimed. The description serves to explain the principles and operations of the claimed subject matter. Other and further features and advantages of the present disclosure will be readily apparent to those skilled in the art upon a reading of the following disclosure.

### DETAILED DESCRIPTION

Reference now will be made in detail to the embodiments of the present disclosure, one or more examples of which are set forth hereinbelow. Each example is provided by way of explanation of the oral electrolyte solution of the present disclosure and is not a limitation. In fact, it will be apparent to those skilled in the art that various modifications and variations can be made to the teachings of the present disclosure without departing from the scope of the disclosure. For instance, features illustrated or described as part of one embodiment, can be used with another embodiment to yield a still further embodiment.

Thus, it is intended that the present disclosure covers such modifications and variations as come within the scope of the appended claims and their equivalents. Other objects, features and aspects of the present disclosure are disclosed in or are apparent from the following detailed description. It is to be understood by one of ordinary skill in the art that the present discussion is a description of exemplary embodiments only and is not intended as limiting the broader aspects of the present disclosure.

The present disclosure relates generally to oral electrolyte solutions comprising lactoferrin. Additionally, the disclosure relates to methods of reducing the duration of diarrhea caused by an infection in a pediatric subject. In other embodiments, the disclosure relates to methods for reducing gastrointestinal irritation and rehydrating a pediatric subject during a bout of diarrhea.

“Nutritional composition” means a substance or formulation that satisfies at least a portion of a subject's nutrient requirements. The terms “nutritional(s)”, “nutritional formula(s)”, “enteral nutritional(s)”, and “nutritional supplement(s)” are used as non-limiting examples of nutritional composition(s) throughout the present disclosure. Moreover, “nutritional composition(s)” may refer to liquids, powders, gels, pastes, solids, concentrates, suspensions, or ready-to-use forms of enteral formulas, oral formulas, formulas for infants, formulas for pediatric subjects, formulas for children, growing-up milks and/or formulas for adults.

“Pediatric subject” means a human less than 13 years of age. In some embodiments, a pediatric subject refers to a human subject that is between birth and 8 years old. In other embodiments, a pediatric subject refers to a human subject between 1 and 6 years of age. In still further embodiments, a pediatric subject refers to a human subject between 6 and 12 years of age. The term “pediatric subject” may refer to infants (preterm or fullterm) and/or children, as described below.

“Infant” means a human subject ranging in age from birth to not more than one year and includes infants from 0 to 12 months corrected age. The phrase “corrected age” means an infant's chronological age minus the amount of time that the infant was born premature. Therefore, the corrected age is the age of the infant if it had been carried to full term. The term infant includes low birth weight infants, very low birth weight infants, and preterm infants. “Preterm” means an infant born before the end of the 37^{th} week of gestation. “Full term” means an infant born after the end of the 37^{th} week of gestation.

“Child” means a subject ranging in age from 12 months to about 13 years. In some embodiments, a child is a subject between the ages of 1 and 12 years old. In other embodiments, the terms “children” or “child” refer to subjects that are between one and about six years old, or between about seven and about 12 years old. In other embodiments, the terms “children” or “child” refer to any range of ages between 12 months and about 13 years.

The oral electrolyte solution of the present disclosure may be substantially free of any optional or selected ingredients described herein, provided that the remaining oral electrolyte solution still contains all of the required ingredients or features described herein. In this context, and unless otherwise specified, the term “substantially free” means that the selected composition may contain less than a functional amount of the optional ingredient, typically less than 0.1% by weight, and also, including zero percent by weight of such optional or selected ingredient.

“Pathogen” means an organism that causes a disease state or pathological syndrome. Examples of pathogens include, but are not limited to bacteria, viruses, parasites, fungi, microbes, or combination(s) thereof.

“Oral electrolyte solution” is a nutritional composition comprising electrolytes that is formulated to facilitate the absorption of water and electrolytes from the intestines. Suitable electrolytes include, but are not limited to sodium, potassium, chloride, calcium, and bicarbonate. An oral electrolyte solution may optionally contain a carbohydrate source.

“Pediatric oral electrolyte solution” is an oral electrolyte solution suitable for administration to pediatric subjects. Generally, a pediatric oral electrolyte solution is formulated with certain concentrations of electrolytes and/or carbohydrates to promote fluid and electrolyte absorption in a pediatric subject.

The term “probiotic” means a microorganism that exerts beneficial effects on the health of the host.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

As used herein, the term “about” should be construed to refer to both of the numbers specified as the endpoint(s) of any range. Any reference to a range should be considered as providing support for any subset within that range.

All percentages, parts and ratios as used herein are by weight of the total formulation, unless otherwise specified.

As used herein, “non-human lactoferrin” means lactoferrin which is produced by or obtained from a source other than human breast milk. In some embodiments, non-human lactoferrin is lactoferrin that has an amino acid sequence that is different than the amino acid sequence of human lactoferrin. In other embodiments, non-human lactoferrin for use in the present disclosure includes human lactoferrin produced by a genetically modified organism. The term “organism”, as used herein, refers to any contiguous living system, such as animal, plant, fungus or micro-organism.

Lactoferrin for use in the present disclosure may be, for example, isolated from the milk of a non-human animal or produced by a genetically modified organism. The oral electrolyte solutions described herein can, in some embodiments comprise non-human lactoferrin, non-human lactoferrin produced by a genetically modified organism and/or human lactoferrin produced by a genetically modified organism.

Suitable non-human lactoferrins for use in the present disclosure include, but are not limited to, those having at least 48% homology with the amino acid sequence of human lactoferrin. For instance, bovine lactoferrin (“bLF”) has an amino acid composition which has about 70% sequence homology to that of human lactoferrin. In some embodiments, the non-human lactoferrin has at least 65% homology with human lactoferrin and in some embodiments, at least 75% homology. Non-human lactoferrins acceptable for use in the present disclosure include, without limitation, bLF, porcine lactoferrin, equine lactoferrin, buffalo lactoferrin, goat lactoferrin, murine lactoferrin and camel lactoferrin.

In some embodiments, the nutritional composition of the present disclosure comprises non-human lactoferrin, for example bLF. bLF is a glycoprotein that belongs to the iron transporter or transferring family. It is isolated from bovine milk, wherein it is found as a component of whey. There are known differences between the amino acid sequence, glycosylation patters and iron-binding capacity in human lactoferrin and bLF. Additionally, there are multiple and sequential processing steps involved in the isolation of bLF from cow's milk that affect the physiochemical properties of the resulting bLF preparation. Human lactoferrin and bLF are also reported to have differences in their abilities to bind the lactoferrin receptor found in the human intestine.

Though not wishing to be bound by this or any other theory, it is believe that bLF that has been isolated from whole milk has less lipopolysaccharide (LPS) initially bound than does bLF that has been isolated from milk powder. Additionally, it is believed that bLF with a low somatic cell count has less initially-bound LPS. A bLF with less initially-bound LPS has more binding sites available on its surface. This is thought to aid bLF in binding to the appropriate location and disrupting the infection process.

bLF suitable for the present disclosure may be produced by any method known in the art. For example, in U.S. Pat. No. 4,791,193, incorporated by reference herein in its entirety, Okonogi et al. discloses a process for producing bovine lactoferrin in high purity. Generally, the process as disclosed includes three steps. Raw milk material is first contacted with a weakly acidic cationic exchanger to absorb lactoferrin followed by the second step where washing takes place to remove nonabsorbed substances. A desorbing step follows where lactoferrin is removed to produce purified bovine lactoferrin. Other methods may include steps as described in U.S. Pat. Nos. 7,368,141, 5,849,885, 5,919,913 and 5,861,491, the disclosures of which are all incorporated by reference in their entirety.

The lactoferrin that is used in certain embodiments may be any lactoferrin isolated from whole milk and/or having a low somatic cell count, wherein “low somatic cell count” refers to a somatic cell count less than 200,000 cells/mL. By way of example, suitable lactoferrin is available from Tatua Co-operative Dairy Co. Ltd., in Morrinsville, New Zealand, from FrieslandCampina Domo in Amersfoort, Netherlands or from Fonterra Co-Operative Group Limited in Auckland, New Zealand.

The oral electrolyte solution may comprise lactoferrin in an amount from about 15 mg/100 mL to about 150 mg/100 mL. In other embodiments lactoferrin is present in an amount from about 30 mg/100 mL to about 110 mg/100 mL. In still other embodiments lactoferrin is present in an amount from about 50 mg/100 mL to about 100 mg/100 mL.

Once the desired form of lactoferrin is obtained, it may be incorporated into the oral electrolyte solution(s) described herein by any method well-known in the art. For example, lactoferrin is a heat labile protein that rapidly undergoes denaturation and loses its bioactivity under the normal thermal processing required in the making of commercially sterile liquids. Therefore in some embodiments lactoferrin may be incorporated into the oral electrolyte solution by aseptically adding lactoferrin solution prepared through sterile filtration. Further lactoferrin may be incorporated into the oral electrolyte solution described herein, by the method according to U.S. Patent Application 2012/0171328 to Banavara et al., incorporated herein in its entirety by reference. However, the present disclosure can also include other processes for incorporating lactoferrin into the oral electrolyte solution disclosed herein.

In some embodiments, where the oral electrolyte solution is a pediatric oral electrolyte solution, lactoferrin may be added to a commercially available pediatric oral electrolyte solution. For example, Enfamil® Enfalyte® (available from Mead Johnson Nutrition Company, Glenview, Ill., U.S.) may be supplemented with the lactoferrin and used in practice of the current disclosure.

The disclosed oral electrolyte solutions described herein can, in some embodiments, also comprise a probiotic. Any probiotic known in the art may be acceptable in this embodiment. In some embodiments, the probiotic may be selected from any Lactobacillus species, Lactobacillus rhamnosus GG (ATCC number 53103), Bifidobacterium species, Bifidobacterium longum BB536 (BL999, ATCC: BAA-999), Bifidobacterium longum AH1206 (NCIMB: 41382), Bifidobacterium breve AH1205 (NCIMB: 41387), Bifidobacterium infantis 35624 (NCIMB: 41003), and Bifidobacterium animalis subsp. lactis BB-12 (DSM No. 10140) or any combination thereof. In a preferred embodiment, the oral electrolyte solution comprises Lactobacillus rhamnosus GG.

If included, the oral electrolyte solution may comprise probiotics from between about 1×10⁵ cfu/100 mL to about 1×10¹⁰ cfu/100 mL. In other embodiments the oral electrolyte solution may comprise between about 1×10⁷ cfu/100 mL to about 1×10⁹ cfu/100 mL.

The probiotic(s) or the present disclosure may be viable or non-viable. As used herein, the term “viable”, refers to live microorganisms. The term “non-viable” or “non-viable probiotic” means non-living probiotic microorganisms, their cellular components and/or metabolites thereof. Such non-viable probiotics may have been heat-killed or otherwise inactivated, but they retain the ability to favorably influence the health of the host. The probiotics useful in the present disclosure may be naturally-occurring, synthetic or developed through the genetic manipulation of organisms, whether such new source is now known or later developed.

The oral electrolyte solutions of the present disclosure may, in some embodiments, optionally comprise vitamin B₃. In some embodiments, vitamin B₃ is present in the oral electrolyte solution in an amount from about 0.5 mg/100 mL to about 3 mg/100 mL solution.

Suitable nonlimiting vitamin B₃ compounds for use in the oral electrolyte solution disclosed herein includes niacin, nicotinic acid, niacinamide, nicotinamide, inisitol nexanicotinate, nicotinyl alcohol, derivatives and salts of any of the foregoing.

Exemplary derivatives of the foregoing vitamin B₃ compounds include nicotinic acid esters, including, but not limited to nicotinyl amino acids, nicotinyl alcohol esters of carboxylic acids, nicotinic acid N-oxide and niacinamide N-oxide. Further suitable esters of nicotinic acid include nicotinic acid esters of C₁-C₂₂, preferable C₁-C₁₆ alcohols. The alcohols are suitably straight-chain or branched-chain, cyclic or acyclic, saturated or unsaturated, aromatic, and substituted or unsubstituted.

Other derivatives of vitamin B₃ suitable for use in the oral electrolyte solutions disclosed herein include derivatives of niacinamide and its derivatives resulting from substitution of one or more of the amide group hydrogens. Examples of derivatives of niacinamide useful herein include nicotinyl amino acids, derived, for example, from the reaction of an activated nicotinic acid compound, such as nicotinic acid azide or nicotinyl chloride, with an amino acid, and nicotinyl alcohol esters of organic carboxylic acids. Specific examples of such derivatives include nicotinuric acid (C₈H₈N₂O₃) and nicotinyl hydroxamic acid (C₆H₆N₂O₂).

Other non-limiting examples of vitamin B₃ compounds useful herein are 2-chloronicotinamide, 6-aminonicotinamide, 6-methylnicotinamide, n-methylnicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, and niaprazine.

In certain embodiments of the oral electrolyte solution(s), salts of the vitamin B₃ compounds may be included. Nonlimiting examples of salts of the vitamin B₃ compound used herein include inorganic salts, such as inorganic salts with anionic inorganic species, such as chloride, bromide, iodide, or carbonate. Other examples of vitamin B₃ organic salts include organic carboxylic acid salts, for example, mono-, di-, and tri-C1-C18 carboxylic acid salts.

In certain embodiments, one or more vitamin B₃ compounds may be included in the oral electrolyte solution. However, in a preferred embodiment the oral electrolyte solution comprises vitamin B₃ as nicotinamide.

Recently, it was discovered in animal studies that nicotinamide, the amide of nicotinic acid, enhanced the killing of the pathogen S. aureus. Without being bound by any particular theory, it is believed that nicotinamide enhances the expression of the myeloid-specific transcription factor, CCAAT/enhancer-binding protein ε C/EBPε above a physiologic level that may lead to the therapeutic killing of S. aureus. Therefore nicotinamide could promote effective immune-mediated clearance of S. aureus. Thus nicotinamide may act as an antimicrobial agent, and can decrease undesirable pathogens in the gut during a bout of diarrhea, thereby reducing the duration of diarrhea symptoms, reducing gastrointestinal irritation, and promoting overall recovery from diarrhea symptoms.

The vitamin B₃ compounds suitable for use in the oral electrolyte solutions described herein are commercially available from a number of sources, including the Sigma Chemical Company (St. Louis, Mo.); ICN Biomedicals, Inc. (Irvine, Calif.); Aldrich Chemical Company (Milwaukee, Wis.); DSM (Evansville, Ind.); and BASF (Florham Park, N.J.).

Additionally, in some embodiments, the oral electrolyte solution may comprise both a probiotic, such as LGG, and vitamin B₃ in addition to lactoferrin.

In still other embodiments, the oral electrolyte solution may comprise zinc. Zinc, as used herein, includes but is not limited to: zinc, zinc oxide, zinc sulfate and mixtures thereof. Zinc also includes all non-limiting exemplary derivatives of zinc compounds including, but not limited to, salts, alkaline salts, esters and chelates of any zinc compound.

Zinc may be present, in some embodiments of the oral electrolyte solution, in an amount from about 0.5 mg/100 mL to about 4 mg/100 mL. In some embodiments, zinc may be present in an amount from about 1 mg/100 mL to about 2.5 mg/100 mL.

In some embodiments, the oral electrolyte solution(s) of the present disclosure may include a source of cultured buttermilk. Without being bound by any particular theory, a source of cultured buttermilk, which includes cultured buttermilk powder, may comprise milk fat globule membrane proteins, short chain fatty acids, lactic cultures and phospholipids that may help in resolving the symptoms of diarrhea.

The source of cultured buttermilk may be included in the oral electrolyte solution in an amount from about 0.5 g/100 mL to about 9 g/100 mL. In some embodiments, the source of cultured buttermilk may be included in an amount from about 1 g/100 mL to about 8 g/100 mL. In some embodiments, the source of cultured buttermilk may be included in an amount from about 3 g/100 mL to about 5 g/100 mL.

The electrolytes included in the oral electrolyte solution may optionally comprise, but are not limited to, sodium, potassium, chloride, calcium, bicarbonate, and mixtures of at least one or more thereof. For example, some embodiments of the oral electrolyte solution may comprise sodium in an amount from about 50 mg/100 mL to about 200 mg/100 mL. In other embodiments, sodium may be present in an amount from about 100 mg/100 mL to about 160 mg/100 mL. In still other embodiments, sodium may be present in an amount from about 110 mg/100 mL to about 150 mg/100 mL.

Potassium may be present in some embodiments of the oral electrolyte solution in an amount from about 60 mg/100 mL solution to about 200 mg/100 mL solution. In other embodiments, potassium may be present in an amount from about 80 mg/100 mL solution to about 150 mg/100 mL solution.

In some embodiments, the oral electrolyte solution may comprise chloride from about 150 mg/100 mL solution to about 275 mg/100 mL solution. In other embodiments chloride is present in an amount from about 180 mg/100 mL solution to about 225 mg/100 mL solution.

The electrolytes included in the oral electrolyte solution may comprise both organic and inorganic salts and their derivatives. Suitable salts for practice of the current disclosure include, but are not limited to, sodium chloride, potassium citrate, sodium citrate and sodium bicarbonate.

The osmolality of the oral electrolyte solution may be from about 100 mOsm/kg water to about 250 mOsm/kg water. In some embodiments, the amounts of sodium, potassium and chloride may vary so long as the overall osmolality of the oral electrolyte solution is within the described range. In still other embodiments, the osmolality may range from about 130 mOsm/kg water to about 180 mOsm/kg water.

Further the osmolarity of the oral electrolyte solution may be from about 100 mOsm/L of solution to about 200 mOsm/L of solution. Additionally, in some embodiments the amounts of sodium, potassium and chloride in the electrolyte solution will vary so long as the overall osmolarity of the oral electrolyte solution is about 100 mOsm/L to about 200 mOsm/L of solution. In some embodiments, the overall osmolarity of the oral electrolyte solution is about 130 mOsm/L to about 180 mOsm/L of solution. Preferably, the osmolar load of the electrolyte solution will facilitate absorption of water and electrolytes from the intestine.

The oral electrolyte solution may further comprise a carbohydrate source. The carbohydrate source may be present in an amount from about 0.5 g/100 mL to about 5 g/100 mL. In other embodiments, the carbohydrate source may be present in an amount from about 1.5 g/100 mL to about 4 g/100 mL. In still other embodiments, the carbohydrate source may be present from about 2.5 g/100 mL to about 3.5 g/100 mL.

Examples of carbohydrate sources include any used in the art, e.g., lactose, glucose, fructose, corn syrup solids, rice syrup solids, maltodextrins, sucrose, starch, rice syrup solids, and the like. In some embodiments, the carbohydrate source includes corn syrup solids, rice syrup solids and/or any other glucose polymers. Moreover, hydrolyzed, partially hydrolyzed, and/or extensively hydrolyzed carbohydrates may be desirable for inclusion in the nutritional composition due to their easy digestibility. Specifically, hydrolyzed carbohydrates are less likely to contain allergenic epitopes. In a preferred embodiment, the carbohydrate source is rice syrup solids.

Carbohydrate materials suitable for use in the present disclosure include hydrolyzed or intact, naturally or chemically modified, starches sourced from corn, tapioca, rice or potato, in waxy or non-waxy forms. Other examples of suitable carbohydrates include various hydrolyzed starches characterized as hydrolyzed cornstarch, maltodextrin, maltose, corn syrup, dextrose, corn syrup solids, rice syrup solids, glucose, and various other glucose polymers and combinations thereof. Examples of other suitable carbohydrates include those often referred to as sucrose, lactose, fructose, high fructose corn syrup, indigestible oligosaccharides such as fructooligosaccharides and combinations thereof.

Further the oral electrolyte solution, in some embodiments, may optionally comprise at least one citrate. A citrate, as used herein, generally refers to the conjugate base of citric acid or to the esters of citric acid. Examples of citrates suitable for use in the nutritional composition include trisodium citrate or triethyl citrate. In some embodiments, trisodium citrate is used in the nutritional composition(s) of the present disclosure as a source of sodium.

The oral electrolyte solutions of the present disclosure may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, honey, imitation pineapple extract, imitation rum extract, imitation strawberry extract, or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

Preservatives may be included in the oral electrolyte solution(s) to extend product shelf life. Suitable preservatives include, but are not limited to, potassium sorbate, sodium sorbate, potassium benzoate, sodium benzoate, calcium disodium EDTA, citric acid, and mixtures thereof.

The oral electrolyte solutions of the present disclosure may provide minimal or partial nutritional support. The oral electrolyte solutions described herein are not formulated to provide total nutritional support, instead are formulated to facilitate the absorption of electrolytes and water from the intestines, especially during an gastrointestinal infection causing diarrhea symptoms. Since, the nutritional composition(s) of the present disclosure are not nutritionally complete, additional breast milk, formula, and/or food should be administered as directed by a physician.

In some embodiments the overall pH of the oral electrolyte solution is from about 4.0 to about 7.2. In some embodiments the overall pH of the oral electrolyte solution is from about 4.8 to about 5.6. Still further, the pH of the oral electrolyte solution may be adjusted according to overall product appearance.

The disclosed oral electrolyte solutions may be provided in liquid form or as a liquid concentrate. The oral electrolyte solutions may, in certain embodiments, comprise a nutritional supplement or children's nutritional product designed for an infant or a pediatric subject. Moreover, the oral electrolyte solution of the present disclosure may be standardized to a specific caloric content, it may be provided as a ready-to-use product, or it may be provided in a concentrated form.

In some embodiments, the electrolyte salts, lactoferrin and carbohydrate source may be provided in powder format and reconstituted in purified water prior to ingestion. If provided in powder format, the particle size is in the range of 5 μm to 1500 μm, more preferably in the range of 10 μm to 300 μm. Further, the electrolyte salts, lactoferrin and carbohydrate source may be provide in tablet, pill, capsule, or any other form that allows for dissolution in purified water.

Some embodiments of the present disclosure are directed to a method for reducing the duration of diarrhea symptoms in a pediatric subject by providing an oral electrolyte solution comprising lactoferrin. The duration of diarrhea is reduced as compared to a pediatric subject who is not provided an oral electrolyte solution comprising lactoferrin. In other embodiments of this method, the nutritional composition may optionally comprise a probiotic, such as Lactobacillus rhamnosus GG and/or vitamin B₃.

Further disclosed herein, is a method for reducing the number of days a pediatric subject suffers from the symptoms of diarrhea comprising providing an oral electrolyte solution comprising lactoferrin and at least one of the following: LGG and vitamin B₃ to a pediatric subject. The reduction in duration of diarrhea symptoms of the pediatric subject of the present method is as compared to a pediatric subject, suffering from the symptoms of diarrhea who is not provided an oral electrolyte solution comprising lactoferrin.

This disclosure also provides a method for reducing gastrointestinal irritation in a pediatric subject suffering from a bout of diarrhea by providing an oral electrolyte solution comprising lactoferrin and at least one of the following: Lactobacillus rhamnosus GG, or vitamin B₃, and mixtures thereof.

Additionally, the present disclosure is directed to a method for rehydrating a pediatric subject during a bout of diarrhea by providing an oral electrolyte solution comprising lactoferrin.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

Formulation examples are provided to illustrate some embodiments of the oral electrolyte solution of the present disclosure but should not be interpreted as any limitation thereon. Other embodiments within the scope of the claims herein will be apparent to one skilled in the art from the consideration of the specification or practice of the oral electrolyte solution or methods disclosed herein. It is intended that the specification, together with the example, be considered to be exemplary only, with the scope and spirit of the disclosure being indicated by the claims which follow the example.

### FORMULATION EXAMPLES

The following formulation examples provide an oral electrolyte solution according to the present disclosure and describes the amount of each ingredient to be included per 100 mL solution.

TABLE 1
Oral electrolyte solution comprising lactoferrin

per 100 mL

| Nutrient | Minimum | Maximum |
|---|---|---|
| Sodium (mg) | 50 | 200 |
| Potassium (mg) | 60 | 200 |
| Chloride (mg) | 150 | 275 |
| Carbohydrate (g) | 0.5 | 5 |
| Lactoferrin (mg) | 25 | 150 |
| LGG (cfu) | 1 × 10⁵ | 1 × 10¹⁰ |
| vitamin B₃ (mg) | 0.5 | 3 |

TABLE 2
Oral electrolyte solution comprising lactoferrin, zinc and a source of
cultured buttermilk

| Nutrient | Amount (g) |
|---|---|
| De-Fluoride Water | 9,600 |
| Rice Solids Clarified | 311 |
| Sodium Chloride 40-60 MESH FCC | 28 |
| Potassium Citrate FCC USP | 28 |
| Flavor Tropical Nat K | 27 |
| Citric Acid Anhydrous FCC USP | 3.5 |
| Lactoferrin | 10 |
| Zinc Oxide | 0.1 |
| Vitamin Premix (Niacin and Vitamin D) | 0.05 |
| Cultured buttermilk powder | 26 |
| LGG | 1.5 |

TABLE 3
Oral electrolyte solution comprising lactoferrin, zinc and a source of
cultured buttermilk

| Nutrient | Amount (g) |
|---|---|
| De-Fluoride Water | 9,600 |
| Rice Solids Clarified | 311 |
| Sodium Chloride 40-60 MESH FCC | 28 |
| Potassium Citrate FCC USP | 28 |
| Flavor Tropical Nat K | 28 |
| Citric Acid Anhydrous FCC USP | 3.4 |
| Lactoferrin | 10 |
| Zinc Oxide | 0.1 |
| Vitamin Premix (Niacin and Vitamin D) | 0.05 |
| Cultured buttermilk powder | 26 |

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. For example, while methods for the production of a commercially sterile liquid nutritional supplement made according to those methods have been exemplified, other uses are contemplated. Therefore, the spirit and scope of the appended claims should not be limited to the description of the preferred versions contained therein.

1. An oral electrolyte solution comprising:
at least one electrolyte selected from the group consisting of sodium, potassium and chloride;
a carbohydrate source; and
from about 15 mg/100 mL to about 150 mg/100 mL lactoferrin,
wherein the oral electrolyte solution has an osmolality from about 100 mOsm/kg water to about 250 mOsm/kg water.

2. The oral electrolyte solution of claim 1, further comprising at least one probiotic.

3. The oral electrolyte solution of claim 2, wherein the probiotic is Lactobacillus rhamnosus GG.

4. The oral electrolyte solution of claim 2, wherein the probiotic is present in an amount from about 1×10⁵ cfu/100 mL to about 1×10¹⁰ cfu/100 mL.

5. The oral electrolyte solution of claim 1 further comprising vitamin B₃.

6. The oral electrolyte solution of claim 1 further comprising zinc.

7. The oral electrolyte solution of claim 1, wherein the oral electrolyte solution is a pediatric oral electrolyte solution.

8. The oral electrolyte solution of claim 1, having an osmolarity of from about 100 mOsm/L to about 200 mOsm/L.

9. The oral electrolyte solution of claim 1, further comprising a source of cultured buttermilk.

10. An oral electrolyte solution, comprising per 100 mL:
(i) between about 50 mg and about 200 mg of sodium;
(ii) between about 60 mg and about 200 mg of potassium;
(iii) between about 150 mg and about 275 mg of chloride;
(iv) between about 0.5 g and about 5 g of a carbohydrate source; and
(iv) between about 15 mg and about 150 mg of lactoferrin.

11. The oral electrolyte solution of claim 10, further comprising about 1 g/100 mL to about 8 g/100 mL of a source of cultured buttermilk.

12. The oral electrolyte solution of claim 10, further comprising 100 mL about 1×10⁵ cfu to about 1×10¹⁰ cfu of Lactobacillus rhamnosus GG.

13. The oral electrolyte solution of claim 10, further comprising about 0.5 mg/100 mL to about 3 mg/100 mL vitamin B₃.

14. A method of reducing the duration of diarrhea in a pediatric subject comprising, administering an oral electrolyte solution comprising at least one electrolyte selected from the group consisting of sodium, potassium and chloride; a carbohydrate source; and lactoferrin to a pediatric subject.

15. The method of claim 14, wherein the lactoferrin is present in an amount from about 15 mg/100 mL to about 150 mg/100 mL.

16. The method of claim 14, wherein the oral electrolyte solution further comprises a probiotic.

17. The method of claim 16, wherein the probiotic comprises Lactobacillus rhamnosus GG.

18. The method of claim 14, wherein the oral electrolyte solution further comprises vitamin B₃.

19. The method of claim 14, wherein the oral electrolyte solution further comprises zinc.

20. The method of claim 14, wherein the oral electrolyte solution further comprises a source of cultured buttermilk.
